(19) United States
(12) Patent Application Publication
(10) Pub. No.: US 2026/0131122 A1
(43) Pub. Date: May 14, 2026

(54) MICRONEEDLE PATCH AND COMPOSITION THEREOF

(71) Applicant: DARWIN PRECISIONS CORPORATION, Hsinchu County (TW)

(72) Inventors: Wan-Ting Wu, Hsinchu County (TW); Yun-Pei Yang, Hsinchu County (TW); Yi-Chen Liu, Hsinchu County (TW)

(21) Appl. No.: 19/315,891

(22) Filed: Sep. 2, 2025

(30) Foreign Application Priority Data
Nov. 14, 2024 (TW) 113143804

(51) Int. Cl. A61M 37/00; A61L 31/04

(52) U.S. Cl. CPC A61M 37/0015; A61L 31/042; A61M 2037/0023

(57) ABSTRACT

A microneedle patch includes a microneedle layer and a carrier layer. The microneedle layer includes a base layer and a plurality of microneedles. The microneedles are formed from a solution containing pectin and hyaluronic acid, are disposed on the base layer, and are spaced apart from each other. The base layer has a first surface and a second surface opposite to each other. The plurality of microneedles is located on the first surface, and the microneedle layer is disposed on the carrier layer via the second surface of the base layer. The present invention also provides a transdermal absorption composition, which contains the pectin and the hyaluronic acid.

### FIELD OF THE INVENTION

The present invention relates to a field of transdermal drug delivery, and more particularly to a microneedle patch and composition thereof.

### BACKGROUND OF THE INVENTION

Microneedle patch (MNP) is a new type of transdermal drug delivery system (TDDS). The microneedles on the patch are quite short and do not touch nerves. Not only do they not cause a pain of subcutaneous injections, but they can also carry biologically active ingredients or drugs through the stratum corneum of the skin and enter the human body. MNP technology can be used in various fields such as aesthetic medicine, medicine, and preventive medicine.

### SUMMARY OF THE INVENTION

The present invention provides a microneedle patch, which helps to improve undesirable conditions in manufacturing processes and improves a yield and a production rate.

The present invention also provides a soluble film having good film forming properties.

The present invention also provides a transdermal absorption composition, which helps to improve the yield and the production rate of microneedle patches, and the film forming properties of soluble films. Moreover, the transdermal absorption composition can provide a sustained release effect of transdermal drug delivery systems.

To achieve one, part, or all of the above purposes or other purposes, one embodiment of the present invention provides a microneedle patch, including a microneedle layer and a carrier layer. The microneedle layer includes a base layer and a plurality of microneedles, wherein the plurality of microneedles are formed by a solution containing pectin and hyaluronic acid and are spaced apart from each other on the base layer. The base layer has a first surface and a second surface opposite to each other, wherein the plurality of microneedles is located on the first surface, and the microneedle layer is disposed on the carrier layer via the second surface of the base layer.

In one embodiment of the present invention, a weight ratio of the pectin to the hyaluronic acid in the solution is from 0.3 to 1.

In order to achieve one, part, or all of the above purposes or other purposes, another embodiment of the present invention also provides a transdermal absorption composition, which contains the pectin and the hyaluronic acid, and a sum of a pectin content and a hyaluronic acid content accounts for 1 to 2 wt % of the transdermal absorption composition, and a weight ratio of the pectin to the hyaluronic acid is from 0.3 to 1.

To achieve one, part, or all of the above purposes or other purposes, another embodiment of the present invention further provides a soluble film prepared from the transdermal absorption composition.

The present invention uses the pectin and the hyaluronic acid as the components of the microneedle layers, so that the microneedle layers with good integrity and flatness can be formed, which is conducive to the formation of the microneedle patches and ensures the yield and the production rate of the microneedle patches.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

The present invention will become more readily apparent to those ordinarily skilled in the art after reviewing the following detailed description and accompanying drawings, in which:

FIG. 1 is a schematic cross-sectional view of a microneedle patch according to an embodiment of the present invention;

FIG. 2 is a schematic flow chart of a method for manufacturing the microneedle patch according to the embodiment of the present invention;

FIGS. 3A to 3C are schematic diagrams showing an implementation of a method for manufacturing the microneedle patch according to a first embodiment of the present invention;

FIG. 4 is a schematic diagram showing an implementation of a method for manufacturing the microneedle patch according to a second embodiment of the present invention;

FIG. 5 is a schematic diagram of a photograph of a microneedle layer of a comparative example of the present invention;

FIG. 6 is a schematic diagram of an hourly dissolution rate of the microneedle layer of the embodiment of the present invention and the comparative example; and

FIG. 7 is a schematic cross-sectional view of a soluble film according to an embodiment of the present invention.

### DETAILED DESCRIPTION OF PREFERRED EMBODIMENTS

The aforementioned and other technical contents, features and effects of the present invention will be clearly presented in the following detailed description of preferred embodiments with reference to the drawings. The direction terms mentioned in the following embodiments only refer to the directions of the attached drawings. Accordingly, the directional terms used are illustrative and not limiting of the present invention. In addition, terms such as “first” and “second” mentioned in this specification or the scope of the patent application are only used to name elements or to distinguish different embodiments or scopes, and are not used to limit the upper or lower limit on the number of elements.

The present invention provides a microneedle patch, which can improve the undesirable conditions in manufacturing processes and increase a yield and a production rate. The microneedle patch provided by the present invention can be used for transdermal drug delivery and transdermal absorption, and thus can be used as a carrier for several biologically active ingredients, pharmaceutically active ingredients, or a combination thereof. The biologically active ingredients are not limited to natural substances, such as plants or extracts thereof, but may also be artificially synthesized substances. In the present invention, a biologically active ingredient means an ingredient that can produce biological effects on humans or other organisms, such as changing human physiological functions and biochemical metabolism. The biologically active ingredients are not limited to those intended to achieve therapeutic or prophylactic effects, but the biologically active ingredients with the therapeutic or prophylactic effects can be equivalent to the pharmaceutical active ingredients. The pharmaceutical active ingredients described in the present invention include natural substances and artificial synthetic substances. The microneedle patch provided by the present invention can further provide a sustained release effect, thereby prolonging a release period of the biologically active ingredients, the pharmaceutically active ingredients, or the combination thereof in the human body or other organisms.

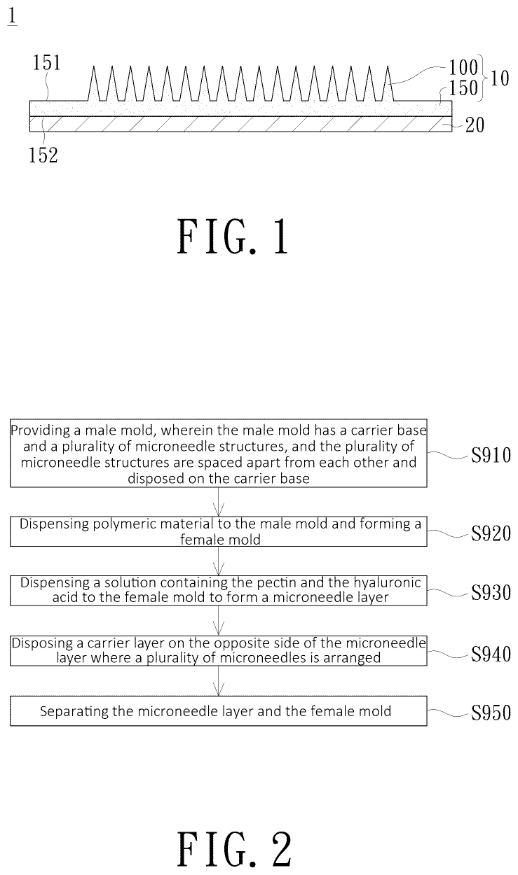

FIG. 1 is a schematic cross-sectional view of a microneedle patch according to an embodiment of the present invention. As shown in FIG. 1, the microneedle patch 1 includes a microneedle layer 10 and a carrier layer 20. The microneedle layer 10 includes a base layer 150 and a plurality of microneedles 100, and the microneedles 100 are spaced apart from each other and disposed on the same side of the base layer 150. The diameter of the microneedles 100 becomes larger as they are closer to the base layer 150. Specifically, the shape of the microneedles 100 can be, for example, a pyramid or a cone. The length of the microneedles 100 can be 100 to 1500 μm, such as 100 μm, 500 μm, 800 μm, 1000 μm, or 1500 μm. The base layer 150 has an appropriate thickness, for example, 5 to 50 μm, and has a first surface 151 and a second surface 152 opposite to each other. The microneedles 100 are disposed on the same surface, such as the first surface 151, and the other surface, such as the second surface 152, can be used to connect the microneedle layer 10 with the carrier layer 20.

In an embodiment of the present invention, the microneedles 100 are formed by a solution containing pectin (INCI: Pectin) and hyaluronic acid (hereinafter referred to as the solution containing the pectin and the hyaluronic acid). It should be noted that the hyaluronic acid includes hyaluronic acid itself and its derivatives. The solution is a water-based solution, and preferably an aqueous solution. The solution containing the pectin and the hyaluronic acid can be injected into a mold and then dried to form the microneedles 100. In a preferred embodiment of the present invention, the base layer 150 is also formed by the solution containing the pectin and the hyaluronic acid. Furthermore, the aqueous solution containing the pectin and the hyaluronic acid can be injected into the mold and then dried to integrally form the microneedle layer 10.

The hyaluronic acid and the pectin are biocompatible polymer compounds. In the present invention, biocompatibility means that a material, a compound, or a composition will not release toxic or harmful substances that cause local or systemic adverse reactions in a human body or other organisms such as inflammatory reactions, immune reactions, and toxic reactions. Therefore, the microneedle layer 10 formed by the solution containing the pectin and the hyaluronic acid can contact the human body without any safety concerns, and can be used as a carrier for transdermal drug delivery and transdermal absorption. In a preferred embodiment of the present invention, due to biocompatibility, the microneedle layer 10 can be degraded over time when implemented in transdermal drug delivery and transdermal absorption, and will not cause adverse reactions in the human body or other organisms. While degrading (hereinafter also referred to as dissolving), the biologically active ingredients and drug active ingredients contained in the microneedles 100 are gradually released into the body to produce effects. The release period of the biologically active ingredients and the active pharmaceutical ingredients may vary depending on the degradation rate of the microneedles 100.

In addition to being biocompatible, the solution containing the hyaluronic acid and the pectin also helps the microneedle layer 10 to maintain its shape, be flat and not curled, and have appropriate elasticity to prevent it from breaking. Since, the microneedle layer 10 has a good shape and structure, the microneedles 100 therein also have a good shape and structure, and can achieve a puncture effect. The flatness and integrity of the microneedle layer 10 are helpful to the yield and the production rate of the microneedle patch 1, for example, ensuring that the carrier layer 20 can be well disposed on the microneedle layer 10.

The hyaluronic acid and the pectin are further prepared in an appropriate ratio into the solution containing the hyaluronic acid and the pectin. The appropriate ratio between the hyaluronic acid and the pectin helps to achieve the aforementioned integrity, smoothness and appropriate elasticity to a higher level. In a preferred embodiment of the present invention, a weight ratio of the pectin to the hyaluronic acid is from 0.3 to 1, for example, 0.3, 0.4, 0.5, 0.6, 0.7, 0.8, 0.9, or 1, and in a more preferred embodiment, it is from 0.3 to 0.7, for example, 0.3, 0.35, 0.4, 0.45, 0.5, 0.55, 0.6, 0.65, or 0.7. To achieve the weight ratio being from 0.3 to 1, for example, an amount of the pectin can be 0.3 to 1 wt %, such as 0.3 wt %, 0.4 wt %, 0.5 wt %, 0.6 wt %, 0.7 wt %, 0.8 wt %, 0.9 wt %, or 1 wt %, when an amount of the hyaluronic acid accounts for 1 wt % of the solution, that is, 1 wt % of a weight of the microneedles 100. A sum of the weights of the pectin and the hyaluronic acid is preferably 1 to 2 wt % of the solution in the embodiment of the present invention, that is, 1 to 2 wt % of a weight of the microneedles 100, for example, 1 wt %, 1.5 wt %, or 2 wt %. In an embodiment of the present invention, a solid content in the solution, such as a total amount of the pectin and the hyaluronic acid, can further affect a thickness of the formed microneedle layer 10, and by adjusting the total amount of the pectin and the hyaluronic acid, the microneedle layer 10 can have an appropriate thickness of 5 to 50 μm, for example, 5 μm, 10 μm, 15 μm, 20 μm, 25 μm, 30 μm, 35 μm, 40 μm, 45 μm, or 50 μm.

The microneedles 100 further contain the biologically active ingredients, the pharmaceutically active ingredients or the combination thereof. The biologically active ingredients can be, for example, vitamin B, vitamin C, salicylic acid, tranexamic acid, ceramide, nicotinamide, ergothioneine, collagen, various plant extracts, or various compounds with aesthetic effects, but are not limited thereto. The active pharmaceutical ingredients can be, for example, an analgesic, an antihistamine, or insulin, but are not limited thereto. Since a degradation rate of the microneedles 100 can affect the release period of the biologically active ingredients and the pharmaceutically active ingredients, the ingredients that are intended to act during a specific period can be further selected. For example, the biologically active ingredients or the active pharmaceutical ingredients that require sustained release can be selected, and the sustained release effect provided by the microneedles 100 can prolong the release and the action period of these ingredients.

The following is an example of a method for manufacturing a microneedle patch 1.

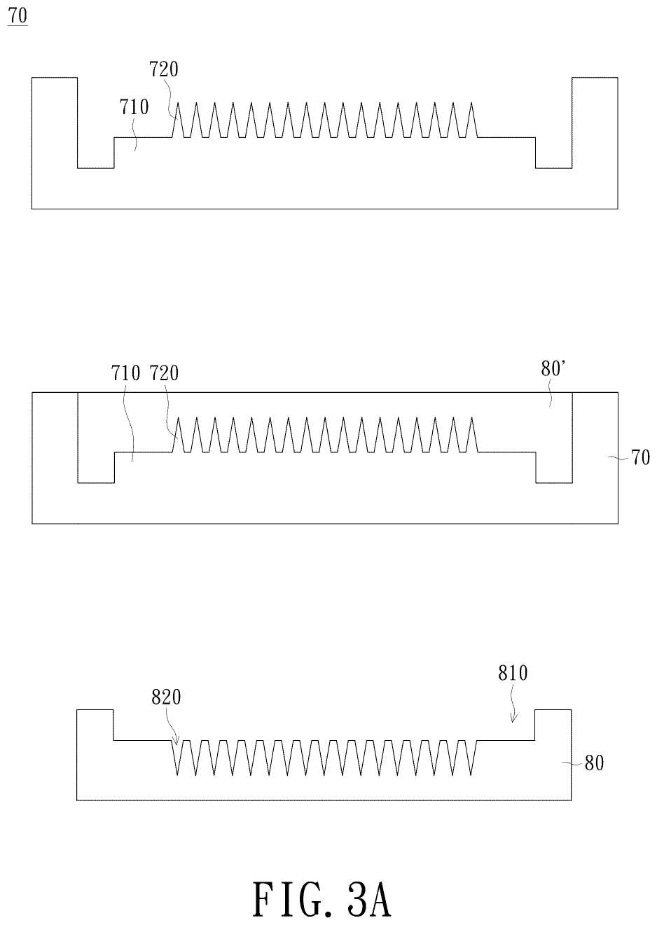

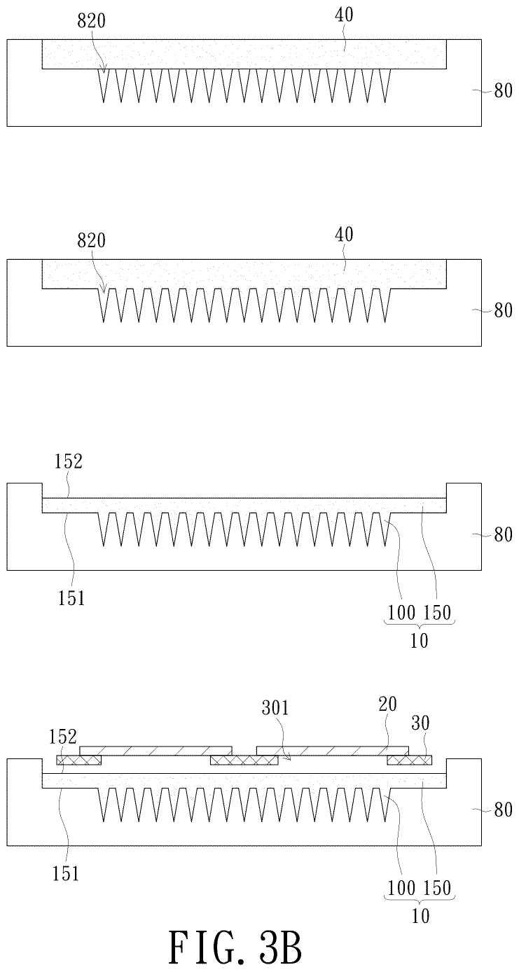

FIG. 2 is a schematic flow chart of a method for manufacturing the microneedle patch according to the embodiment of the present invention. As shown in FIG. 2 and FIG. 3A to FIG. 3C, the processes include steps S910 to S950. Step S910 includes: providing a male mold, wherein the male mold has a carrier base and a plurality of microneedle structures, and the plurality of microneedle structures are spaced apart from each other and disposed on the carrier base. Step S920 includes: dispensing polymeric material to the male mold and forming a female mold. Step S930 includes: dispensing a solution containing the pectin and the hyaluronic acid to the female mold to form a microneedle layer. Step S940 includes: disposing a carrier layer on the opposite side of the microneedle layer where a plurality of microneedles is arranged. Step S950 includes: separating the microneedle layer and the female mold.

The male mold 70 of step S910 is a metal mold. The embodiment of the present invention does not limit a material of the male mold 70, but for example, it can be titanium, copper, aluminum, nickel, tungsten, stainless steel, titanium alloy, nickel alloy, aluminum alloy, or copper alloy. As shown in FIG. 3A, a plurality of microneedle structures 720 are spaced apart from each other and are disposed on a carrier base 710 at an appropriate distribution density. A shape of the microneedle structures 720 may be, for example, a cone, a triangular pyramid, or a quadrangular pyramid, and a height can be, for example, 100 to 1500 μm. In addition, a distance between adjacent microneedle structures 720 can be, for example, between 0.5 and 5 mm. A polymeric material 80′ in step S920 can be, for example, polyethylene, polypropylene, polylactic acid, polybutylene succinate, or polydimethylsiloxane, but is not limited thereto. As shown in FIG. 3A, step S920 further includes: distributing the polymeric material 80′ to the carrier base 710 and the plurality of microneedle structures 720 thereon, so that a plurality of tapered holes 820 are formed in the female mold 80. A shape of the tapered hole 820 corresponds to the shape of the microneedles 100.

As shown in FIG. 3B, step S930 may further include: preparing a solution 40 containing the pectin and the hyaluronic acid. Since the microneedle layer 10 formed by the solution 40 containing the pectin and the hyaluronic acid can be used as a carrier for transdermal drug delivery and transdermal absorption, the solution 40 containing the pectin and the hyaluronic acid can also be called a transdermal absorption composition. The preparation of the solution 40 containing the pectin and the hyaluronic acid mainly includes weighing appropriate amounts of a solvent, the pectin and the hyaluronic acid, and then dissolving the pectin and the hyaluronic acid in the solvent. The solvent used is preferably water. The purity of water is preferably distilled water or above, and more preferably double distilled water (ddH₂O). The pectin and the hyaluronic acid can generally be mixed in water in powder form, wherein a weight ratio of the pectin powder to the hyaluronic acid powder is preferably from 0.3 to 1, such as 0.3, 0.4, 0.5, 0.6, 0.7, 0.8, 0.9, or 1. In an embodiment of the present invention, the hyaluronic acid with a molecular weight less than 800,000 Da may further be used, such as 800,000 Da, 500,000 Da, 100,000 Da, 80,000 Da, 50,000 Da, 10,000 Da, 8,000 Da, 5,000 Da, or 1,000 Da. In some embodiments, the molecular weight of the hyaluronic acid may be between 3,000 Da and 800,000 Da. The hyaluronic acid of different molecular weights can be used in combination to prepare the solution, or the hyaluronic acid of a single molecular weight can be used in the preparation of the solution.

In an embodiment of the present invention, the total weight of the pectin and the hyaluronic acid is preferably 1 to 2 wt % of the solution, for example, 1 wt %, 1.5 wt %, or 2 wt %. Pectin and hyaluronic acid aqueous solutions are generally viscous. The viscosity can be, for example, between 15 cps and 5000 cps, such as 15 cps, 50 cps, 100 cps, 250 cps, 500 cps, 1000 cps, 2000 cps, 3000 cps, 4000 cps, and 5000 cps, but is not limited thereto. In principle, as long as the formation of the microneedle layer 10 is not hindered, the embodiment of the present invention has no limitation on the viscosity. Step S930 also includes a bubble removal procedure. The bubble removal procedure is usually carried out after dispensing the solution containing the pectin and the hyaluronic acid. During this procedure, air bubbles in the solution are removed to prevent holes from being generated in the microneedle layer 10. The bubble removal procedure can be carried out naturally by a weight of the solution, or it can be accelerated by other means such as vibration and heat. The aforementioned viscosity does not hinder the formation of the microneedle layer 10; for example, it cannot hinder the effective removing of bubbles.

Step S930 may further include: adding the biologically active ingredients, the active pharmacological ingredients or the combination thereof to the solution containing the pectin and the hyaluronic acid. The definitions and examples of biologically active ingredients and active pharmacological ingredients are as described above and no redundant detail is to be given herein. The amount of the biologically active ingredients, active pharmacological ingredients or the combination thereof added can be adjusted depending on, for example, its dosage requirement, the desired release period, and the degradation period of the carrier. For example, an analgesic can be added in an amount ranging from several milligrams to several hundred milligrams. In some embodiments of the present invention, the solution containing the pectin and the hyaluronic acid added with the biologically active ingredients, the active pharmacological ingredients or the combination thereof can be first distributed to the tapered holes 820 of the female mold 80, and then the solution containing the pectin and the hyaluronic acid without the biologically active ingredients, the active pharmacological ingredients or the combination thereof can be distributed to the space 810 above the tapered holes 820, so that the formed microneedle layer 10 contains the biologically active ingredients, the active pharmacological ingredients or the combination thereof only in the microneedles 100.

Step S930 also includes: performing a drying procedure. The drying procedure usually follows the bubble removal procedure. The drying procedure can form a film composed of the pectin and the hyaluronic acid, namely the microneedle layer 10. The drying procedure can be carried out naturally at normal temperature and humidity, or can be carried out at specific temperature and humidity, for example, by using a number of means such as a baking device, a drying device, and a heating device. In some embodiments of the present invention, drying can be performed by using the baking device at an appropriate temperature. As shown in FIG. 3B, a volume of the film formed after the drying procedure, especially the base layer 150, can be reduced compared to a volume of the solution, but this is a normal phenomenon. In addition, the microneedle layer 10 shall be presented with a complete shape. For example, when the female mold 80 is substantially in a shape of a rectangular parallelepiped with specific length and width, the microneedle layer 10 shall have equivalent length and width in the same direction. In addition, the microneedle layer 10 shall be flat and not curled, and preferably have appropriate elasticity without breaking.

As described further herein, a ratio of the pectin to the hyaluronic acid in the embodiment of the present invention helps to ensure that the microneedle layer 10 is intact in shape, flat and not curled, and has appropriate elasticity without breaking. Furthermore, under the same drying conditions, the film 10 formed by the solution with a weight ratio of the pectin to the hyaluronic acid being from 0.3 to 1 can better meet the requirements of complete shape and flatness without curling. The complete and flat film 10, i.e., the integrity and flatness of the microneedle layer 10, is helpful for the subsequent steps. The integrity and flatness of the microneedle layer 10 can ensure that the shape and structure of the microneedles 100 are symmetrical and complete without any deflection or breaking, and can achieve the puncture effect. The present invention verifies the relationship between the weight ratio of the pectin to the hyaluronic acid and film forming properties. The results are shown in Table 1 below:

TABLE 1

| | Aqueous | | | | | | | |
|---|---|---|---|---|---|---|---|---|
| | solution of | | | | | Integrity | | |
| | hyaluronic | | Pectin | | Pectin/hyaluronic | (water | | Film |
| No. | acid (wt %) | | (wt %) | | acid weight ratio | repellency) | Flatness | status |
| 1 | 0.5 | wt % | 0 | wt % | 0 | repellent | curl | won't |
| | | | | | | | | break |
| 2 | 0.6 | wt % | 0 | wt % | 0 | repellent | curl | won't |
| | | | | | | | | break |
| 3 | 0.85 | wt % | 0 | wt % | 0 | repellent | curl | won't |
| | | | | | | | | break |
| 4 | 1 | wt % | 0 | wt % | 0 | repellent | curl | won't |
| | | | | | | | | break |
| 5 | 1 | wt % | 0.2 | wt % | 0.2 | slightly | slightly | won't |
| | | | | | | repellent | curl | break |
| 6 | 1 | wt % | 0.3 | wt % | 0.3 | non-repellent | make | won't |
| | | | | | | | level | break |
| 7 | 1 | wt % | 0.4 | wt % | 0.4 | non-repellent | make | won't |
| | | | | | | | level | break |
| 8 | 1 | wt % | 0.5 | wt % | 0.5 | non-repellent | make | Won't |
| | | | | | | | level | break |
| 9 | 1 | wt % | 0.6 | wt % | 0.6 | non-repellent | make | won't |
| | | | | | | | level | break |
| 10 | 1 | wt % | 0.7 | wt % | 0.7 | non-repellent | make | won't |
| | | | | | | | level | break |
| 11 | 1 | wt % | 0.8 | wt % | 0.8 | non-repellent | make | resilient |
| | | | | | | | level | |
| 12 | 1 | wt % | 0.9 | wt % | 0.9 | non-repellent | make | too soft |
| | | | | | | | level | |
| 13 | 1 | wt % | 1 | wt % | 1 | non-repellent | make | too soft |

level

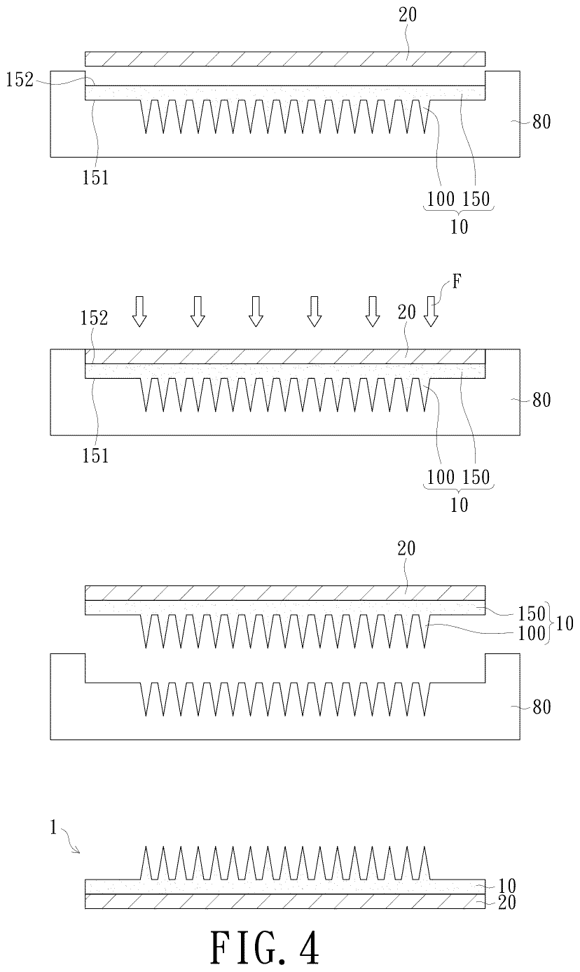

In Table 1, Nos. 1 to 4 are comparative examples, which use solutions containing the pectin and the hyaluronic acid with different weight percentages to form a film 10′; Nos. 5 to 13 are examples, which use solutions containing the pectin and the hyaluronic acid with different pectin/hyaluronic acid weight ratios to form the film 10, wherein the solutions of Nos. 1 to 4 do not contain the pectin. The results show that the film 10 formed by the solution with a weight ratio of the pectin to the hyaluronic acid being from 0.3 to 1 has good integrity and flatness, and the film formed by the solution with a weight ratio being from 0.3 to 0.7 has more appropriate elasticity and does not break. In contrast, as shown in Table 1 and the comparative examples in FIG. 5, the film 10′ formed by the solution with a weight ratio other than being from 0.3 to 1 is incomplete (water-repellent), curled, and causes the microneedles 100′ to be skewed or broken.

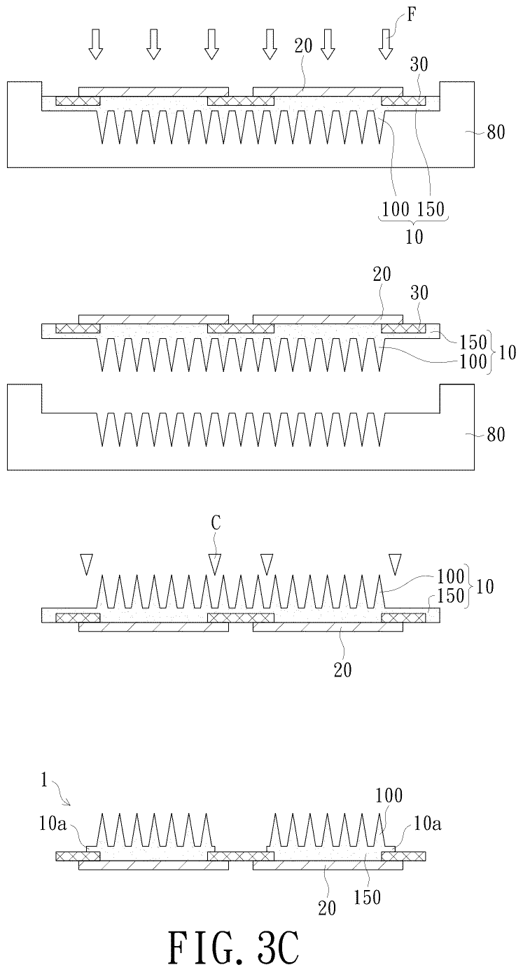

As shown in FIG. 3B, step S940 further includes: disposing a release film 30 and the carrier layer 20 on the microneedle layer 10 on the opposite side of the microneedles 100, such as the second surface 152 of the base layer 150. The release film 30 and the carrier layer 20 can be sequentially disposed on the base layer 150, or can be combined first and then disposed on the base layer 150 together. In the embodiment of the present invention, the release film 30 is disposed between the carrier layer 20 and the base layer 150, and generally has a plurality of hollow areas 301. The release film 30 and the hollow areas 301 thereof are helpful for subsequently dividing the microneedle layer 10 into a plurality of parts, but the present invention is not limited thereto. As shown in FIG. 4, the carrier layer 20 can also be directly disposed on the base layer 150 without the release film 30. A material of the carrier layer 20 can be a synthetic or natural fabric, a synthetic or natural polymer, such as woven, non-woven, or knitted fabrics. The carrier layer 20 is preferably compliant and flexible so as to be able to be evenly attached to the surface of the biological body. For example, the carrier layer 20 can be a film or a cloth. The material of the carrier layer 20 can also be selected based on certain functional requirements such as waterproof, breathable, antibacterial, odor, aesthetics, and hand feel. Additionally, the material of the carrier layer 20 can also be selected based on medical consumables that are available in the market such as bandages, tapes, and patches.

As shown in FIGS. 3C and 4, step S940 can further include: applying force F to the carrier layer 20 toward the microneedle layer 10 to promote bonding between the carrier layer 20 and the microneedle layer 10. Step S950 can further include: removing the microneedle layer 10 bonded to the carrier layer 20 from the female mold 80, thereby completing the manufacture of the microneedle patch 1. In addition, in some embodiments of the present invention, the microneedle layer 10 can further be cut according to the shape of each hollow area 301 to divide the microneedle layer 10 into a plurality of microneedle layers 10a. Cutting can be performed using a cutting tool C. The cutting tool C can be, for example but not limited to, a punch die or a laser cutter.

As described above, the microneedle patch 1 of the embodiment of the present invention can provide the sustained release effect, wherein due to the biocompatibility of the microneedle layer 10, the microneedles 100 can be degraded over time, and the biologically active ingredients and the pharmaceutically active ingredients carried therein can be released within a specific period of time based on the degradation rate. The present invention also tests the relationship between the ingredients of the microneedle layer 10 and its degradation rate (hereinafter also referred to as dissolution rate), and finds the relationship between the weight ratio of the pectin to the hyaluronic acid and the dissolution rate. The results are shown in Table 2 below:

TABLE 2

| | Aqueous | | | | Pectin/ | | |
|---|---|---|---|---|---|---|---|
| | solution of | | | | hyaluronic | | |
| | hyaluronic | | Pectin | | acid weight | 4-hour | 6-hour |
| No. | acid (wt %) | | (wt %) | | ratio | solubility | solubility |
| 1 | 0.5 | wt % | 0 | wt % | 0 | 100% | 100% |
| 2 | 0.6 | wt % | 0 | wt % | 0 | 100% | 100% |
| 3 | 0.85 | wt % | 0 | wt % | 0 | 100% | 100% |
| 4 | 1 | wt % | 0 | wt % | 0 | 100% | 100% |
| 5 | 1 | wt % | 0.2 | wt % | 0.2 | 80% | 100% |
| 6 | 1 | wt % | 0.3 | wt % | 0.3 | 40% | 75% |
| 7 | 1 | wt % | 0.4 | wt % | 0.4 | 40% | 60% |
| 8 | 1 | wt % | 0.5 | wt % | 0.5 | 35% | 60% |
| 9 | 1 | wt % | 0.6 | wt % | 0.6 | 35% | 60% |
| 10 | 1 | wt % | 0.7 | wt % | 0.7 | 35% | 60% |
| 11 | 1 | wt % | 0.8 | wt % | 0.8 | 35% | 60% |
| 12 | 1 | wt % | 0.9 | wt % | 0.9 | 35% | 60% |
| 13 | 1 | wt % | 1 | wt % | 1 | 35% | 60% |

Table 2 shows that within the same period, for example, 4 hours and 6 hours, the dissolution rates of the films 10 of Nos. 5 to 13 are slower than the dissolution rates of the films 10′ of Nos. 1 to 4, wherein the solutions of Nos. 1 to 4 do not contain pectin. It can be seen from this that pectin should help provide the sustained release effect and extend the release period of the biologically active ingredients, the pharmaceutically active ingredients or the combination thereof. As shown in Table 2, the dissolution rates of the films 10 formed by the solutions with the weight ratios of the pectin to the hyaluronic acid greater than 0.2 (the films 10 of Nos. 6 to 13) did not reach 100% after 6 hours, indicating that it can make the release period of the biologically active ingredients, the pharmaceutically active ingredients or the combination thereof exceed 6 hours, thereby providing a slower release effect.

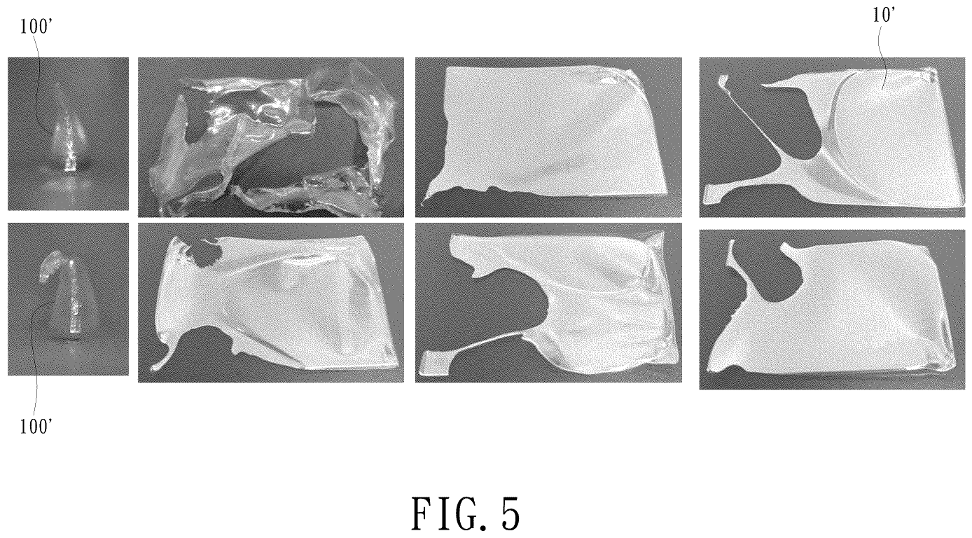

The present invention further compares the degradation rate of the microneedles in the microneedle patch 1 of the embodiment with a commercially available microneedle patch. As described above, the microneedle layer 10 of the microneedle patch 1 contains the pectin and the hyaluronic acid in the weight ratio being from 0.3 to 1, while the commercially available microneedle patch does not contain pectin. The test method is to apply the microneedle patch 1 of the present invention and the commercially available microneedle patch in groups of different individuals (n=3), then collect the microneedle patch 1 and the commercially available microneedle patch from time to time, and calculate the degradation rates of the microneedles 100 of the embodiment of the present invention and the commercially available microneedle patch. As shown in FIG. 6, the square points in each hour are respectively the degradation rates of the microneedles 100 of the microneedle patches 1 of the embodiment applying to individuals 1, 2, and 3 after the indicated hour. The solid line curve is obtained by averaging each hour, which reflects the hourly dissolution rate of the microneedles 100. The round dots in each hour represent the degradation rates of the microneedles of the commercially available microneedle patch applying to individuals 1, 2, and 3 after that hour, and the dotted curve is obtained by averaging the degradation rates. As shown in FIG. 6, the microneedles 100 of the embodiment of the present invention degrade at a nearly stable rate from the 1st to the 6th hours, and the dissolution rate has not yet reached 100%. In contrast, the degradation of the commercially available microneedle patches is completed after 4 hours at the latest, reflecting that the biologically active ingredients and pharmaceutically active ingredients cannot be released continuously after 4 hours.

The present invention also provides a soluble film, which is prepared from the aforementioned transdermal absorption composition. As mentioned above, the transdermal absorption composition is the solution containing the pectin and the hyaluronic acid, and further contains the biologically active ingredients, the active pharmacological ingredients or a combination thereof. Since the materials, compounds or compositions used to prepare the soluble film are biocompatible, the soluble film is endowed with solubility. The solubility can also be called degradability.

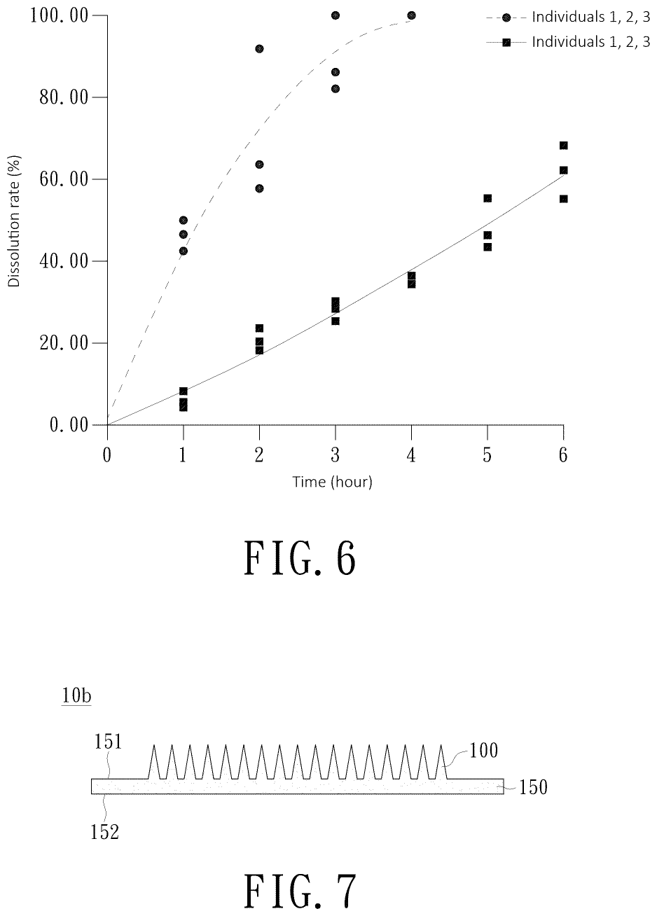

In an embodiment of the present invention, as shown in FIG. 7, a soluble film 10b can further include the base layer 150 and the plurality of microneedles 100, and the microneedles 100 are spaced apart from each other and disposed on the same side of the base layer 150. One side of the base layer 150 on which the plurality of microneedles 100 is disposed is referred to herein as the first surface 151. The soluble film 10b can be used as a microneedle film, or can be used to make the microneedle patch 1 as described above. In a preferred embodiment of the present invention, the solution of the pectin and the hyaluronic acid used to prepare the soluble film 10b has the pectin/hyaluronic acid weight ratio being from 0.3 to 1, so it has good film forming properties: the shape is complete, flat and not curled, and has appropriate elasticity without breaking. Both the microneedle film and the microneedle patch 1 can be used as transdermal drug delivery devices.

The degradation of the soluble film 10b of the embodiment of the present invention preferably occurs under appropriate conditions. Furthermore, the soluble film 10b preferably degrades under conditions above a room temperature. The room temperature is preferably a temperature below 35° C., and more preferably below 30° C., such as 20° C. to 30° C. A degradable (dissolvable) temperature of the soluble film 10b is preferably above 35° C. For example, when the soluble film 10b is used as the microneedle film or made into the microneedle patch 1 and applied to an organism, the body temperature of the organism can degrade the microneedles 100 to release the biologically active ingredients, the active pharmacological ingredients or the combination thereof. In some embodiments of the present invention, the microneedles 100 can be completely degraded at a temperature higher than room temperature, such as 35° C. to 40° C. within 8 to 10 hours. In a preferred embodiment of the present invention, the degradation degree of the microneedles 100 is less than 50% within 4 hours at an appropriate temperature, such as 35° C. to 40° C. and thus the microneedles 100 can be further used as a sustained release transdermal drug delivery device.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A microneedle patch comprising a microneedle layer and a carrier layer, wherein:
the microneedle layer comprises a base layer and a plurality of microneedles, wherein the microneedles are formed by a solution containing pectin and hyaluronic acid, and the microneedles are spaced apart from each other on the base layer; and
the base layer has a first surface and a second surface opposite to each other, wherein the microneedles are located on the first surface, and the microneedle layer is arranged on the carrier layer via the second surface of the base layer.

2. The microneedle patch according to claim 1, wherein a weight ratio of the pectin to the hyaluronic acid in the solution is from 0.3 to 1.

3. The microneedle patch according to claim 2, wherein the weight ratio of the pectin to the hyaluronic acid in the solution is from 0.3 to 0.7.

4. The microneedle patch according to claim 1, wherein a sum of a pectin content and a hyaluronic acid content in the solution accounts for 1 to 2 wt % of the solution.

5. The microneedle patch according to claim 1, wherein a molecular weight of the hyaluronic acid in the solution is less than or equal to 800,000 Da.

6. The microneedle patch according to claim 5, wherein the molecular weight of the hyaluronic acid in the solution further is 3,000 to 800,000 Da.

7. The microneedle patch according to claim 5, wherein the base layer is formed by the solution containing the pectin and the hyaluronic acid, and the microneedle layer further is integrally formed by the solution containing the pectin and the hyaluronic acid.

8. The microneedle patch according to claim 1, wherein the microneedles further contain biologically active ingredients, active pharmaceutical ingredients, or a combination thereof.

9. A transdermal absorption composition comprising pectin and hyaluronic acid, wherein a sum of a pectin content and a hyaluronic acid content accounts for 1 to 2 wt % of the transdermal absorption composition, and a weight ratio of the pectin to the hyaluronic acid is from 0.3 to 1.

10. The transdermal absorption composition according to claim 9, further comprising biologically active ingredients, active pharmaceutical ingredients, or a combination thereof.

11. A soluble film prepared from the transdermal absorption composition according to claim 9.

12. The soluble film according to claim 11, further comprising a base layer and a plurality of microneedles, wherein the microneedles are spaced apart from each other on the base layer.
